(19) United States
(12) Patent Application Publication
(10) Pub. No.: US 2010/0003290 A1
(43) Pub. Date: Jan. 7, 2010

(54) OIL AND WATER REPELLENT COSMETIC POWDER AND METHODS OF MAKING AND USING SAME

(75) Inventors: David SCHLOSSMAN, Short Hills, NJ (US); Yun SHAO, Piscataway, NJ (US)

Correspondence Address: THOMPSON HINE L.L.P.;Intellectual Property Group, P.O. BOX 8801, DAYTON, OH, 45401-8801, US

(73) Assignee: KOBO PRODUCTS, INC., South Plainfield, NJ (US)

(21) Appl. No.: 12/239,788

(22) Filed: Sep. 28, 2008

(51) Int. Cl. A61K 8/02; A61Q 90/00

(52) U.S. Cl. 424/401; 427/2.14

(57) ABSTRACT

Disclosed is a coated powder that comprises a powder substrate having a coating thereon that includes a 2-(perfluoroalkyl)ethyl alcohol phosphate of the formula

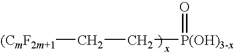

wherein m is about 4 to 6 and x is 1 to 3. Also disclosed herein is a composition, such as a cosmetic composition, that includes the coated powder and a process for rendering a powder hydrophobic and lipophobic by treating the powder with a composition including the 2-(perfluoroalkyl)ethyl alcohol phosphate of the above formula.

### BACKGROUND

The present application relates generally to oil and water repellent powders (i.e., hydrophobic and lipophobic powders), particularly those used in cosmetic compositions. The oil and water repellent powders disclosed herein relate to novel 2-(perfluoroalkyl)ether alcohol phosphate-treated powders, a method for producing such treated powders, and cosmetic formulations comprising such treated powders.

Insoluble powder materials in aqueous or organic media, for example colorful pigments, sunscreen agents, talc and the like, are commonly employed in the cosmetics and other industries, such as the paint, coatings and plastics industries, to serve a variety of purposes. The insoluble cosmetic powders may also include many quite different materials such as metal oxides, metal silicates, other inorganic salts, pigment extenders or fillers such as talc and silica as well as organic materials such as lakes, which are organic dyes fixed on metallic salts, and other materials, as is well known in the art. Additional examples of pigments that can be employed in the present invention are set forth herein below. The powders will be described herein as it applies to cosmetic powders, with the understanding that the novel materials, methods and compositions of matter may be useful in other industries where such powders may be employed, as will be apparent to those skilled in the art from this disclosure.

Many pigment materials, for example metal oxides and carbonates, have a somewhat hydrophilic surface, which renders the powder particles prone to agglomerate in cosmetics and have poor skin feel. Accordingly, over the years, many compositions and methods have been employed to surface treat cosmetic powders, in order to overcome these and other problems. Polyfluoroalkyl phosphates (a phosphate ester) have been used to coat cosmetic powders, to render the pigment hydrophobic and lipophobic. Because the treated powder repels skin oil, the resultant color cosmetics can resist damage due to skin oil as well as moisture. The extended wear has been well accepted by consumers. Fluorosilanes have also been used, but these compounds are expensive and as such are unpractical for most formulations. Additionally, fluorosilane coated powders require a high level of coating to achieve the same level of water and oil repellency that the disclosed novel coated powders achieve.

Many of the coatings or treatments mentioned above have met with considerable commercial success; however, the coatings may include long fluoroalkyl chains during production of which perfluorooctanoic acid (PFOA) is a by-product or the coating will break down thermally to generate PFOA. PFOA has been found to be persistent in the environment and has been detected in wildlife and humans in low levels. Testing of this compound has indicated that it may have effects of concern for humans, animals, or the environment. Current grades of polyfluoroalkyl phosphates under the INCI names C9-15 fluoroalcohol Phosphate and DEA C8-18 Perfluoroalkylethyl Phosphate contain some level of PFOA and/or may break down to give rise to PFOA.

While the current polyfluoroalkyl phosphate(s) used to coat cosmetic powders results in hydrophobic and lipophobic coated powders, which are beneficial to repel skin oil and water for cosmetics that can resist damage due to skin oil and moisture, the safety concern of PFOA out weighs the benefits. The cosmetic industry needs a pigment coating that provides the same beneficial hydrophobic and lipophobic properties to the coated powder without PFOA as a by product of the coating process or as a degradation product.

### SUMMARY

Disclosed herein is a coated powder that has a coating that is free of PFOA and will not produce PFOA as a degradation product. The coated powder has hydrophobic and lipophobic properties as a result of being coated with a coating or a composition that includes a 2-(perfluoroalkyl)ethyl alcohol phosphate of the formula

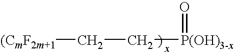

on a powder substrate, wherein m is about 4 to 6 and x is 1 to 3.

In another aspect the coated powder is incorporated into a composition such as a cosmetic composition. The cosmetic composition may be liquid or dry make-up such as foundation or pressed powder, lipstick, blush, eyeshadow, or mascara. Additionally, the cosmetic composition may be anhydrous or an emulsion.

Also disclosed herein is a process for rendering a powder hydrophobic and lipophobic. The process comprises providing a powder and treating the powder with a composition containing a 2-(perfluoroalkyl)ethyl alcohol phosphate of the above formula.

The powder substrate may be any suitable cosmetic powder such as inorganic and organic pigments and fillers; talc; mica; sericite; kaolin; starches; barium sulfate; calcium carbonate; porous or non-porous silica in various shapes including spherical, ellipsoidal, irregular, rod and other known shapes; hydroxyapatite; and hollow or solid polymeric powders or microspheres of polymethylmethacrylate, polyvinylidene chloride copolymer, polyethylene, cellulose or nylon or other suitable polymer. Preferably the cosmetic powder is an inorganic pigment or filler, such as titanium dioxide and/or zinc oxide.

### DETAILED DESCRIPTION

Disclosed is a coated powder that is hydrophobic and lipophobic for use in cosmetic compositions. In particular, the novel coated powders are especially but not exclusively cosmetic powders, including pigments, lakes of organic colorant and filler. Also disclosed is a process for rendering the powder hydrophobic and lipophobic and the coated powders resulting therefrom. This disclosure extends to products that incorporate the coated powders, particularly novel cosmetic compositions in which the coated powders can be applied.

The hydrophobic and lipophobic properties of the coated powder make the powder especially attractive to formulators in the cosmetics industry enabling the novel coated powders to be specified for a wide range of applications without undue concern as to the nature of the liquid phase into which the powder is to be dispersed. Furthermore, the coating disclosed herein is suitable for a wide range of cosmetic powders including many or most of the powders used in everyday cosmetic products such as liquid or powder makeup, lipstick, nail enamel, eye shadow, mascara and so on. Thus the formulator may freely specify cosmetic powders having the disclosed coating(s) to meet an exceptionally diversified range of cosmetics requirements.

These desirable properties can be obtained by treating any one or more of a wide range of cosmetic powders with a 2-(perfluoroalkyl)ethyl Alcohol Phosphate or a composition thereof to ultimately form a coated powder. The 2-(perfluoroalkyl)ethyl Alcohol Phosphate has a structure illustrated by the following Formula 1

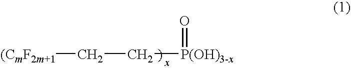

where m is about 4 to 6 and x is 1 to 3. With the alkyl group as a butyl, pentyl or hexyl the chain length is short enough that perfluorooctanoic acid (PFOA) is not introduced from the precursor(s) used in forming the compound or formed if the compound breaks down. In Examples 1-3, below, the 2-(perfluoroalkyl)ethyl Alcohol Phosphate is 2-(perfluorohexyl)ethyl alcohol phosphate (m=6 and X=1). The 2-(perfluoroalkyl)ethyl Alcohol Phosphate coating may include any suitable additives that are customary employed in cosmetic powder or pigment coating processes, if desired.

Cosmetic Powders

Some suitable cosmetic or other powders that can be employed in this invention include: inorganic and organic pigments and fillers; talc; mica; sericite; kaolin; starches; barium sulfate; calcium carbonate; porous or non-porous silica in various shapes including spherical, ellipsoidal, irregular, rod and other known shapes; hydroxyapatite; and hollow or solid polymeric powders or microspheres of polymethylmethacrylate, polyvinylidene chloride copolymer, polyethylene, cellulose or nylon or other suitable polymer. Other suitable cosmetic or other powders will be known or be or become apparent to those skilled in the art.

The powders employed as substrates in the processes of the invention may have any desired regular or irregular shape including spherical or ball like particles with irregular porous surfaces, needles, rods, flakes, rhomboids and so on. In one embodiment, the powders of interest may be finely divided particles which are intended to be uniformly dispersed in the finished product. Fine particle size and uniformity of dispersion are desirable characteristics that contribute to the quality of the finished product and to efficient utilization of the powder. Finer powder particles expose more surface area of particle material in the end product, enabling the particles' color or other property to be more efficiently imparted to the finished product. Uniform dispersion of the particles in liquid or even powder excipients is desirable or even essential to provide a consistent commercial product with good shelf life which is free of discoloration, settling or other blemishes.

Some suitable inorganic pigments which may benefit from the coatings of the present invention include: titanium dioxide; zinc oxide; iron oxide; alumina oxide; chromium oxide; mango violet; ultramarines, composites of metal oxides or of a metal oxide and an inorganic salt and any other inorganic pigment powder useful in the cosmetic or other relevant arts. If desired, prior to the coating treatment, powders such as titanium dioxide, zinc oxide and other inorganic pigments or fillers, may be treated with silica, alumina, boron nitride or other known inorganic coatings, singly or in combinations.

Some suitable organic pigments include aluminum, barium, calcium, and zirconium lakes of FD&C and D&C grades of Red No. 6, Red No. 7, Red 21, Red No. 27 and Yellow No. 5. Other suitable inorganic or organic pigments will be known or be or become apparent to those skilled in the art.

Preferably, the reactants and reaction conditions employed in treating the powder with the 2-(perfluoroalkyl)ethyl Alcohol Phosphate are selected to provide covalent bonding to metal oxide, hydroxide, carbonate, silicate or other reactive moieties on the surfaces of the cosmetic powder particles. However, ionic, hydrogen or van der Waals bonding in addition to, or in the alternative, may also provide satisfactory bonding between the coating and the substrate powder particle.

Particle Size

There is no particular limitation as to the particle size of the powders employed in the invention. However, a mean particle size in the range of from about 0.01 to about 100 micron is preferred and a mean particle size in the range of from about 0.01 to about 20 micron is more preferred. Desirably, at least about 90 percent, preferably at least about 98 percent, and more preferably at least about 99.5 percent of the particles lie within the preferred average particle size range. Some preferred powders for use in the invention are free of oversize particles that may impart grittiness and are also free of overly fine particles whose presence may be undesirable in the processes of the invention described herein.

Treating the Powder

The 2-(perfluoroalkyl)ethyl Alcohol Phosphate coating may be applied to the powder using a variety of techniques know to one of skill in the art. The powder(s) can be treated with a suitable coating agent in a liquid medium, for example by mixing or spraying the coating agent with or on to the powder. Another method may involve making an aqueous slurry of a 2-(perfluoroalkyl)ethyl Alcohol Phosphate salt in a base solution, like diethanolamine, ammonia, or sodium hydroxide for example, where the pH is adjusted during the coating process to liberate the 2-(perfluoroalkyl)ethyl Alcohol Phosphate to coat the pigments. Subsequently, the mixture may be dried by heating to remove the solvents, optionally under vacuum to remove volatile solvents, if employed. The mixture may be heated at a temperature of about 30° C. to about 150° C. to remove the solvent and dry the powder. Other suitable times and temperatures will be known to those of skill in the art, having regard to the materials employed, or can be determined without undue experimentation. Optionally the drying may be conducted under vacuum. After drying the coated powder, the powder may be milled or pulverized using, for example, a jet mill, hammer mill, or other suitable mill. Powders treated by mixing the powder with a solution containing the 2-(perfluoroalkyl)ethyl Alcohol Phosphate is preferred, as illustrated below in Examples 1-3, but the process is not limited thereto.

The solution containing the 2-(perfluoroalkyl)ethyl Alcohol Phosphate may contain one or more suitable solvents, i.e., a solvent capable of dissolving the respective coating agent or composition. The 2-(perfluoroalkyl)ethyl Alcohol Phosphate is soluble in polar solvents. The solvent may promote even distribution of the 2-(perfluoroalkyl)ethyl Alcohol Phosphate over the surface of the substrate powders and may readily evaporate upon heating to facilitate the drying step, if included in the process. This may be advantageous in the treating process as the process may include drying the coated powder after mixing. For example, a volatile organic solvent may be used, such as isopropyl alcohol, acetone may be employed for the 2-(perfluoroalkyl)ethyl Alcohol Phosphate.

The 2-(perfluoroalkyl)ethyl Alcohol Phosphate coating may also include a hydrophobic coating agent. The hydrophobic coating agent may be applied to the powder by treating the powder with the hydrophobic coating agent or a composition thereof before treating the powder with the 2-(perfluoroalkyl)ethyl Alcohol Phosphate. This process may be referred to as a two-stage process because the coatings are applied one at a time in sequence. Preferably, in the first stage the hydrophobic coating agent is applied to the powder and the resultant coated powder is dried. The hydrophobic coating agent coat may be applied as a solution wherein the solvent is a hydrocarbon, such as hexane or any other solvent capable of dissolving the hydrophobic coating agent. The solvent may also be sufficiently volatile to be removed by drying, such as isopropyl alcohol, ethanol, or the like. The powder may be treated with the solution containing the hydrophobic coating agent by mixing the powder into the solution, by spraying the powder with the solution, or any other method known to one of skill in the art. The treated powder may then be dried by heating to remove the solvent and further heated for about 1 to about 10 hours at a temperature of about 30° C. to about 150° C. In the second stage, the dried treated powder having the hydrophobic coating is then treated with a 2-(perfluoroalkyl)ethyl Alcohol Phosphate coating or composition as described above. The resultant coated powder is hydrophobic and lipophobic and is free of PFOA. The tests used to determine that the coated powder is hydrophobic and lipophobic are explained below in Examples 1-3.

Alternately, the hydrophobic coating agent may be part of the 2-(perfluoroalkyl)ethyl Alcohol Phosphate composition that is used to treat the powder. Here the powder may be treated with a mixture or composition containing 2-(perfluoroalkyl)ethyl Alcohol Phosphate and the hydrophobic coating agent in isopropyl alcohol. The two coating agents are preferably dissolved in the isopropyl alcohol and then mixed with the powder. Thereafter the entire mixture is heated to remove the solvent and further heated, as explained above, to dry the treated powder. The resultant coated powder is hydrophobic and lipophobic and is free of PFOA. The tests used to determine that the coated powder is hydrophobic and lipophobic are explained below in Examples 1-3. For any of the embodiments disclosed herein the process may also include milling the treated powder to remove any agglomerates or overly sized coated powders.

In an embodiment that includes the hydrophobic coating agent, the quantity or proportion of 2-(perfluoroalkyl)ethyl Alcohol Phosphate to the hydrophobic coating agent may be varied according to the nature of the powder substrate, the solvent used in the treatment process and other desired cosmetic properties for the coated powder such as smooth feel and good adhesion to the skin. The ratio of coating agent 2-(perfluoroalkyl)ethyl Alcohol Phosphate to the hydrophobic coating agent may be from 10:1 to 1:10, but preferably 5:1 to 1:3.

The hydrophobic coating agent may be any such agent known to one of skill in the art. Preferred hydrophobic coating agents are chemically reactive with the substrate powder to provide a durable covalent bond thereto and have hydrophobic chemical backbones or substituents that can provide a hydrophobic outer layer, skin or shell around each individual powder particle. The coating agent may react, for example, with hydroxyl groups, oxide ions, available oxygen atoms or other suitable reactive groups normally present on the surface of the cosmetic powder being coated.

The hydrophobic coating agent may be, for example, a silicone, a silane, a metal soap, a titanate, an organic wax, and mixtures thereof. With respect to the silicone, the hydrophobic coating may be a methicone, a dimethicone, their copolymers or mixtures thereof. The silicone may also be an organosilicon compound, for example dimethylpolysiloxanes having a backbone of repeating—Me2SiO—units (“Me” is methyl, CH₃), methyl hydrogen polysiloxanes having a backbone of repeating—MeHSiO—units and alkoxysilanes of formula RₙOSiH(₄₋ₙ) where “R” is alkyl and “n” is the integer 1, 2 or 3. With respect to the silane, the hydrophobic coating agent may be an alkoxysilanes, for example an alkyltriethoxy or an alkyltrimethoxy silanes available from OSI Specialities or PCR. The alkoxysilane may be a triethoxycaprylylsilane or a perfluoroalkylethyl triethoxysilane having a C3 to C12 alkyl group that is straight or branched. One such alkoxysilane is Dynasylan® OCTEO available from Degussa AG. With respect to the metal soap, the hydrophobic coating agent may be a metal myristate, metal stearate, a metal palmitate, a metal laurate or other fatty acid derivatives known to one of skill in the art. The metal, for example, may be magnesium or aluminum. With respect to the titanate, the hydrophobic coating agent may be an organotitanate as taught in U.S. Pat. No. 4,877,604 to Mitchell Schlossman (“Schlossman '604” hereinafter), the disclosure of which is herein incorporated by reference thereto. Schlossman '604 discloses isopropyl titanium triisostearate as one preferred coating agent. With respect to the organic wax, the hydrophobic coating agent may be a synthetic wax like polyethylene or a natural wax like carnauba wax.

Depending upon which hydrophobic coating agent is employed a suitable solvent is needed. A suitable aprotic solvent can be employed for the functionalized silane or other silicon compound. Also, if desired the silane, can be solubilized in a volatile organic solvent such as isopropyl alcohol, heptane, isoheptane, isooctane, isononane and petroleum distillates such as those available from Phillips Chemical under the trade names or trademarks Soltrol 130, Soltrol 150 and Soltrol 170. Another useful solvent for functionalized silicon compounds is an isopar solvent. Isopar solvents are a range of solvents each comprising a high-purity, fractionated partially neutralized mixture of isoparaffinic acids which are available in different grades such as isopar C, which comprises C7-C8 solvents, isopar E or isopar G. Water or other suitable solvent may be employed for the organotitanate as described in Schlossman '604.

The coated powder made according to any of the processes disclosed herein may have a quantity of coating relative to the powder substrate that is generally at least 0.1 percent of the coated product. If the amount is over about 30 percent by weight, the coated powder may be too wet and may tend to agglomerate unacceptably. Thus, in one embodiment the quantity is in a range of from about 0.1 to about 30 percent by weight based on the weight of the coated powder, preferably from about 1 to about 10 percent and more preferably from about 2 to about 5 percent of the weight of the coated powder.

In another aspect, any of the coated powders, described above, having a coating that includes a 2-(perfluoroalkyl)ethyl Alcohol Phosphate may be incorporated into a cosmetic composition. Some examples of cosmetic compositions in which the treated powders may be employed include liquid or dry make-ups such as foundation or pressed powder, lipsticks, blushes, eyeshadow, and mascara. Examples 4-6 list the ingredients and the process for preparing, respectively, a liquid foundation composition, an eyeshadow composition, and a dual dry/wet powder. The novel coated powders disclosed herein are beneficial to cosmetic compositions in that the powders are hydrophobic, lipophobic, disperse well and may be incorporated into anhydrous or emulsion based cosmetic compositions, which makes the powders very versatile. The emulsion may be an oil-in-water, water-in-oil, or a water-in-silicon emulsion.

Many other products are known to those skilled in the art that may benefit from such a versatile coated powder. The coated powders, for instance, may be incorporated into other industrial products where the particle material is customarily used and where hydrophobic and lipophobic properties are beneficial, for example paints and plastics.

### Treating the Powder

### Example 1

A 10% solution of 2-(perfluorohexyl)ethyl Alcohol Phosphate in isopropyl alcohol was prepared and 50 g of this solution was mixed with 100 g of Titanium dioxide (Tipaque PF-671 available from Kobo Products). The mixture was heated to remove the solvent and was further heated at 100° C. for three hours to dry the powder. The dry powder was then milled to remove any large lumps.

### Example 2

For Example 2 100 g of Titanium dioxide (Tipaque PF-671 available from Kobo Products) was mixed with 2 g of Triethoxycaprylylsilane and dissolved in hexane. The mixture was heated to remove solvent and further heated at 110° C. for three hours to dry the powder. At this stage of the process the dry powder obtained is hydrophobic. The dry hydrophobic powder is then blended with 50 g of a 10% solution of 2-(perfluorohexyl)ethyl Alcohol Phosphate in isopropyl alcohol. The mixture was heated to remove the solvent and further heated at 110° C. for three hours to dry the powder. The dry powder was then milled to remove any large lumps.

### Example 3

Here 3 g of 2-(perfluorohexyl)ethyl Alcohol Phosphate and 2 g of Perfluorooctylethyl triethyoxysilane were dissolved in isopropyl alcohol. The resultant solution was mixed with 100 g of Titanium dioxide (Tipaque PF-671 available from Kobo Products). The mixture was heated to remove the solvent and further heated at 100° C. for three hours to dry the powder. The dry powder was then milled to remove any large lumps.

### Example 4

In this example, 5 g of 2-(perfluorohexyl)ethyl Alcohol Phosphate was mixed with 50 g of diethanolamine (DEA) aqueous solution at about 60° C. to about 80° C. while mixing. The amount of DEA is such that the final pH is between 8 to 11. The resulting DEA based slurry is translucent.

Separately, 100 g of Titanium dioxide (Tipaque PF-671 available from Kobo Products) was dispersed in 1 L of water. The DEA based slurry was added into the dispersed TiO₂ and mixed thoroughly. Then, 1N HCl was added to adjust the pH to a range of 3-5. The mixture was agitated for an additional hour and filtered to remove the solvent. The powder was washed with 100 ml of distilled water three times and the powder sludge was collected and heated at 100° C. for three hours to dry the powder. The dry powder was then milled to remove any large lumps.

### Test for Hydrophobicity and Lipophobicity

Examples 1-4 were then tested to determine if they were hydrophobic and lipophobic as well. The Hydrophobic Test includes placing about 1 g of the dry treated powder into a 2 oz. jar filled with water, shaking the jar, then letting the jar stand for about 2-about 5 minutes. If the powder settles out or floats and the water appears clear, then the test indicates that the dry treated powder is hydrophobic. The Lipophobic Test includes placing about 1 g of the dry treated powder into a 2 oz. jar filled with mineral oil. If the powder floats, then the test indicates that the dry treated powder is lipophobic and if the powder floats for more than 1 hr, then the lipophobicity is satisfactory.

TABLE 1

| | Hydrophobicity | Lipophobicity |
|---|---|---|
| Example | water phase | powder |
| 1 | clear* | floats** |
| 2 | clear* | floats** |
| 3 | clear* | floats** |

*hydrophobic
**lipophobic

### Cosmetic Compositions

The coated powders disclosed herein may be incorporated in to compositions, such as cosmetic compositions. While the coated powders are hydrophobic and lipophobic, the powders can be dispersed in a liquid where sufficient silicone fluids are present. The lipophobic property of the coated powder results in a powder that is able to repel skin oil, which is more polar than other oils, such as silicone fluids. So while the coated powders repel skin oil, the powders are still dispersible to form a composition.

### Example 5

### Long Lasting Liquid Foundation

The following ingredients are employed in the proportions indicated to prepare an oil-in-water liquid makeup. The metal oxides, talc, ultramarine blue, and nylon-12 are each listed “w/coating,” which in this example indicates that the powders were coated with 2-(perfluorohexyl)ethyl Alcohol Phosphate according to the procedure described in Example 1 above. The pigments or powders that are “w/coating” in Table 2 below may alternately be coated according to either procedure described in Examples 2-4.

TABLE 2

| | % |
|---|---|
| Part A | |
| Titanium dioxide (w/coating) | 9.3 |
| Red iron oxide (w/coating) | 0.6 |
| Yellow iron oxide (w/coating) | 1.0 |
| Black iron oxide (w/coating) | 0.025 |
| Talc (w/coating) | 4.0 |
| Ultramarine Blue (w/coating) | 0.072 |
| Nylon-12 (spherical w/coating) | 2.0 |
| Part B | |
| Stearic acid | 1.5 |
| PEG-40 Stearate | 2.0 |
| Squalane | 5.0 |
| Cetyl alcohol | 0.5 |
| Dimethicone | 2.0 |
| C12-15 Alkyl Benzoate | 5.0 |
| Part C | |
| Water | 56.43 |
| Polysorbate 20 | 0.1 |
| Propylene Glycol | 8.0 |
| Tetrasodium EDTA | 0.1 |
| Methyl Paraben | 0.4 |
| Hectorite | 1.0 |
| Xanthan Gum | 0.2 |
| Triethanolamine, 99% | 0.8 |

The ingredients of Part A without Nylon-12 are ground together and then Nylon-12 is added and mixed well. Separately, the ingredients of Part B are mixed and heated to 50° C. until homogeneous. Then the mixture of Part A is added to the mixture of Part B and mixed well to form mixture AB. Separately, the ingredients of Part C are mixed and heated to 50° C. until homogeneous. Next, mixture AB is slowly added to the mixture of Part C with mixing for 3 minutes to form the liquid foundation. The liquid foundation is then cooled to 35° C. The liquid foundation containing the coated powders disclosed herein is waterproof and oilproof.

### Example 6

### Eyeshadow

The following ingredients are employed in the proportions indicated to prepare a fluoroalcohol-treated eyeshadow. The metal oxides, talc, ultramarine blue, and nylon-12 are each listed “w/coating,” which in this example indicates that the powders were coated with 2-(perfluorohexyl)ethyl Alcohol Phosphate according to the procedure described in Example 1 above. The pigments or powders that are “w/coating” in Table 2 below may alternately be coated according to either procedure described in Examples 2-4.

TABLE 3

| | % |
|---|---|
| Phase 1 | |
| Talc (w/coating) | 21.85 |
| Mica (w/coating) | 7.60 |
| Ultramarine Blue (w/Coating) | 5.32 |
| Black iron oxide (w/coating) | 0.07 |
| Magnesium Myristate | 16.62 |
| Polyethylene | 3.82 |
| HDI/Trimethylol Hexyllactone Crosspolymer (and) Silica | 3.82 |
| Phase 2 | |
| Dimethicone | 9.87 |
| (and)Trimethylsiloxysilicate | |
| Phenoxyethanol (and) Methylparaben (and) | 0.77 |
| Propylparaben (and) Ethylparaben | |
| Phase 3 | |
| Mica (and) Titanium Dioxide (and) Triethoxycaprylylsilane | 9.42 |
| Mica (and) Iron Oxide (C.I. 77491) (and)Triethoxycaprylylsilane | 2.68 |
| Phase 4 | |
| Mica (and) Titanium Dioxide (and) Tin Oxide | 15.11 |

The ingredients of Phase 1 are ground together using a Waring blender or Osterizer for two minutes to develop the color and disperse the powder ingredients. The binder ingredients of Phase 2 were added to Phase 1 drop wise while grinding at high speed for two minutes to aid complete distribution of the liquid to coat the powders. Then, the Phase 3 color pearls were added to the mixture and blended at high speed for two minutes to mix the pearl materials and coat them with the binder as well. Next, Phase 4 was added and ground for one minute only to blend in the shimmer particles. Thereafter the resulting eyeshadow was pressed into appropriate pans at about 900-1000 psi.

### Example 7

### Dual Dry/Wet Powder

The following ingredients are employed in the proportions indicated to prepare dual dry/wet powder. The metal oxides, talc, ultramarine blue, and nylon-12 are each listed “w/coating,” which in this example indicates that the powders were coated with 5% 2-(perfluorohexyl)ethyl Alcohol Phosphate and 2% of Triethoxycaprylylsilane according to the procedure described in Example 2 above. The pigments or powders that are “w/coating” in Table 2 below may alternately be coated according to either procedure described in Examples 1, 3, or 4.

TABLE 4

| | % |
|---|---|
| Part A | |
| Kaolin | 32.00 |
| Mica | 22.61 |
| Mica (w/coating) | 22.42 |
| Titanium dioxide (w/coating) | 3.00 |
| Red iron oxide (w/coating) | 0.55 |
| Yellow iron oxide (w/coating) | 0.77 |
| Black iron oxide (w/coating) | 0.10 |
| Nylon-12 | 1.0 |
| Propylparaben | 0.10 |
| Methylparaben | 0.20 |
| Part B | |
| Hydrogenated Polycyclopentadiene | 3.0 |
| (and) Squalane (Kobogaurd 5400SQ) | |
| Squalane | 12.25 |
| Sorbitan Oleate | 1.00 |
| Dimethicone (350 C.) | 1.00 |

The ingredients of Part A were placed into a Waring Blender under a fume hood and mixed for 2-3 minutes at high speed. The ingredients of Part B were mixed together separately and then slowly added to the Phase A mixture in the Waring Blender, still under a fume hood, and mixed 2-3 minutes at high speed. The resulting cosmetic powder was passed through a #20 mesh screen and pressed into appropriate pans at about 1000 psi.

The resulting cosmetic compositions of Examples 4-6 are free of PFOA and have hydrophobic and lipophobic properties that contribute to the cosmetic's overall waterproof and oilproof characteristics because they include at least one of the novel coated powders disclosed herein.

While illustrative embodiments have been described above, it is, of course, understood that various modifications will be apparent to those of ordinary skill in the art. Many such modifications are contemplated as being within the spirit and scope of the following claims.

What is claimed is:

1. A coated powder comprising a powder substrate having a coating thereon that includes a 2-(perfluoroalkyl)ethyl alcohol phosphate of the formula 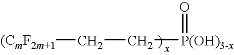
wherein m is about 4 to 6 and x is 1 to 3.

2. The coated powder of claim 1 wherein m is 6 and x is 1.

3. The coated powder of claim 1 wherein the powder substrate is selected from the group consisting of an organic pigment, an inorganic pigment, a filler, a mineral, a synthetic powder, and combinations thereof.

4. The coated powder of claim 3 wherein the powder substrate has an average particle size of not more than about 100 microns.

5. The coated powder of claim 3 wherein the powder substrate is an inorganic pigment that includes at least one of titanium dioxide, zinc oxide and alumina.

6. The coated powder of claim 1 having hydrophobic and lipophobic properties.

7. The coated powder of claim 1 wherein the coating further includes a hydrophobic coating agent.

8. The coated powder of claim 7 wherein the hydrophobic coating agent is selected from the group consisting of a silicone, a silane, a metal soap, a titanate, an organic wax, and combinations thereof.

9. The coated powder of claim 8 wherein the hydrophobic coating agent is a silicone.

10. The coated powder of claim 10 wherein the silicone is a methicone, a dimethicone, copolymers thereof, or mixtures thereof.

11. The coated powder of claim 8 wherein the hydrophobic coating agent is a silane.

12. The coated powder of claim 11 wherein the silane is a triethoxycaprylysilane, a perfluoroalkylethyl triethoxysilane, or mixtures thereof.

13. The coated powder of claim 12 wherein the silane is a perfluoroalkylethyl triethoxysilane having a C3 to C12 alkyl group.

14. The coated powder of claim 13 wherein the perfluoroalkylethyl triethoxysilane has a C8 alkyl group.

15. The coated powder of claim 1 wherein the coated powder is free of perfluorooctanoic acid.

16. A composition comprising:
a powder treated with a 2-(perfluoroalkyl)ethyl alcohol phosphate of the formula 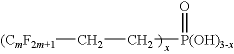
where m is about 4 to 6 and x is 1 to 3 to modify the surface of the powder.

17. The composition of claim 16 wherein m is 6 and x is 1.

18. The composition of claim 16 wherein the powder comprises one or more powders selected from the group consisting of an organic pigment, an inorganic pigment, a filler, a mineral, and a synthetic powder.

19. The composition of claim 18 wherein the powder is an inorganic pigment including at least one of titanium dioxide, zinc oxide, and alumina.

20. The composition of claim 16 wherein the modified surface of the powder further includes a hydrophobic coating agent.

21. The composition of claim 20 wherein the hydrophobic coating agent is selected from the group consisting of a silicone, a silane, a metal soap, a titanate, an organic wax, and mixtures thereof.

22. The composition of claim 21 wherein the hydrophobic coating agent is a silicone.

23. The composition of claim 22 wherein the silicone is a methicone, a dimethicone, copolymers thereof, or mixtures thereof.

24. The composition of claim 21 wherein the hydrophobic coating agent is a silane.

25. The composition of claim 24 wherein the silane is a triethoxycaprylysilane, a perfluoroalkylethyl triethoxysilane, or mixtures thereof.

26. The composition of claim 24 wherein the silane is a perfluoroalkylethyl triethoxysilane having a C1 to C12 alkyl group.

27. The composition of claim 26 wherein the perfluoroalkylethyl triethoxysilane has a C8 alkyl group.

28. The composition of claim 16 wherein the composition is a cosmetic composition.

29. The composition of claim 28 wherein the cosmetic composition is anhydrous.

30. The composition of claim 29 wherein the anhydrous composition is a lipstick or a pressed powder.

31. The composition of claim 28 wherein the composition is an oil/water emulsion, a water/oil emulsion, or a water/silicone emulsion.

32. The composition of claim 28 further comprising a biocompatible excipient.

33. The composition of claim 16 wherein the treated powder has hydrophobic and lipophobic properties

34. The composition of claim 16 wherein the composition is free of perfluorooctanoic acid.

35. A process comprising:
a) providing a powder;
b) treating the powder with a composition containing a 2-(perfluoroalkyl)ethyl alcohol phosphate having the formula 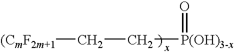
where m is about 4 to 6 and x is 1 to 3, the process rendering the powder hydrophobic and lipophobic.

36. The process of claim 35 wherein treating the powder includes mixing the powder with the composition to form a mixture.

37. The process of claim 36 further comprising drying the powder and optionally milling the dry powder.

38. The process of claim 35 wherein the composition is an isopropyl alcohol solution.

39. The process of claim 35 wherein the powder is one or more cosmetic powders selected from the group consisting of an organic pigment, an inorganic pigment, a filler, a mineral, and a synthetic powder.

40. The process of claim 35 further comprising treating the powder with a hydrophobic coating agent.

41. The process of claim 40 wherein the treating with the hydrophobic coating agent is before the treating with the 2-(perfluoroalkyl)ethyl alcohol phosphate.

42. The process of claim 40 wherein the hydrophobic coating agent is selected from the group consisting of a silicone, a silane, a metal soap, a titanate, an organic wax, and mixtures thereof.

43. The process of claim 40 wherein treating with the hydrophobic coating agent includes mixing a hydrophobic coating agent with the powder.

44. The process of claim 40 further comprising drying the powder.

45. The process of claim 35 wherein the composition further includes a hydrophobic coating agent.

46. The process of claim 45 wherein the hydrophobic coating agent is selected from the group consisting of a silicone, a silane, a metal soap, a titanate, an organic wax, and mixtures thereof.

47. A coated powder prepared in accordance with the method of claim 35.

48. A cosmetic composition including a coated powder prepared in accordance with the method of claim 35.
